(19) United States
(12) Patent Application Publication
(10) Pub. No.: US 2006/0198789 A1
(43) Pub. Date: Sep. 7, 2006

(54) Target validation assay

(76) Inventors: Zhigang Weng, Brookline, MA (US); Jeno Gyuris, Winchester, MA (US); Steven C. Clark, Winchester, MA (US)

Correspondence Address: FISH & NEAVE IP GROUP;ROPES & GRAY LLP, 1251 AVENUE OF THE AMERICAS FL C3, NEW YORK, NY, 10020-1105, US

(21) Appl. No.: 11/326,795

(22) Filed: Jan. 6, 2006

Related U.S. Application Data
(60) Provisional application No. 60/642,243, filed on Jan. 6, 2005.

(51) Int. Cl. A61K 49/00; C12Q 1/68; C12N 5/08; C12N 15/87

(52) U.S. Cl. 424/9.2; 435/6; 435/455; 435/366

(57) ABSTRACT

An method of determining whether a gene of interest is necessary for a tumor cell to maintain its tumorigenicity is disclosed. The method is useful for validation of cancer therapeutic targets in vivo, using shRNAs and tumor xenografts. The inducible shRNA method operates an in vivo RNAi competition assay.

### CROSS-REFERENCE TO RELATED APPLICATION

This application claims the benefit of U.S. provisional patent application No. 60/642,243, filed Jan. 6, 2005, the disclosure of which is incorporated herein by reference in its entirety.

### FIELD OF THE INVENTION

The field of the invention is molecular biology and oncology.

### BACKGROUND OF THE INVENTION

Once a cancer therapeutic target is identified, the target has to be validated. In principle, RNA interference (RNAi) is a valuable tool in target validation studies because it allows rapid assessment of the effects of stringently reducing the expression of a target gene. Nevertheless, application of RNAi for target validation in vivo presents significant challenges. Except for specialized local administration, e.g., intraocular administration, effective delivery of small inhibitory RNA (siRNA) in vivo, remains problematic. And while production of transgenic mice engineered to express inducible short hairpin RNA (shRNA) might yield valuable target validation information, the time and expense required for separate production of transgenic animals for each of the hundreds of targets considered in a typical target discovery research program would be impractical. There is a need for new developments in the practical application of RNAi technology in target validation.

### SUMMARY OF THE INVENTION

The invention provides an shRNA-based in vivo method of target validation. The method includes the steps of: (a) providing a first subpopulation of cells of a given tumor-forming cell line, wherein the subpopulation is engineered to express an shRNA against a first gene of interest, in response to an inducer; (b) providing one or more additional subpopulations of cells of the same cell line, wherein each subpopulation is engineered to express an shRNA in response to the inducer; (c) injecting into each of at least two immuno-compromised mice a mixture of cells representing the first subpopulation of cells and each of the one or more additional subpopulations of cells; (d) allowing time for tumors to develop in the mice from the injected cells; (e) administering an effective amount of the inducer to at least one mouse, thereby establishing an shRNA expression group, while withholding the inducer from at least one mouse, thereby establishing an uninduced group; (f) harvesting the tumors after a suitable time period; and (g) determining the relative representation of the cells engineered to express the shRNA against each gene of interest in the shRNA expression group and in the uninduced group.

In preferred embodiments of the invention, the tumor-forming cell line is a human cell line, e.g., HCT-116, DLD-1, HT-1080, HCT-15, A-549, SW 620, LNCAP, 22Rv1, DU145, or PC-3. Preferably, at least one of the additional subpopulations of cells is a subpopulation of cells engineered to express at least one control shRNA, e.g., a negative control, a positive control, or both. In some embodiments of the invention, one or more of the additional subpopulations of cells is engineered to express an shRNA against at least one additional gene of interest. In some embodiments of the invention, one or more of the additional subpopulations of cells is engineered to express an shRNA against at least two additional genes of interest. In some embodiments of the invention, one or more of the additional subpopulations of cells is engineered to express an shRNA against at least five additional genes of interest. In some embodiments of the invention, one or more of the additional subpopulations of cells is engineered to express an shRNA against at least ten additional genes of interest.

Preferably, the mixture of the first subpopulation of cells and the one or more additional subpopulations of cells injected into each mouse consists of 10³ to 10⁸ cells. More preferably, the mixture of the first subpopulation of cells and the one or more additional subpopulations of cells injected into each mouse consists of 10⁵ to 10⁷ cells. Often, the mixture of the first subpopulation of cells and the one or more additional subpopulations of cells injected into each mouse consists of approximately 10⁶ cells. After harvesting the tumors, the relative representation of the cells can be measured by quantitative PCR analysis.

Unless otherwise defined, all technical and scientific terms used herein have the same meaning as commonly understood by a person skilled in the art to which the invention pertains. In case of conflict, the present specification, including definitions, will control. All publications, patents and other references mentioned herein are incorporated by reference in their entirety.

Throughout this specification and claims, “comprise,” in all its forms such as “comprises” and “comprising,” is intended to include the stated integer or group of integers, but not to exclude any other integer or group of integers.

Although methods and materials similar or equivalent to those described herein can be used in the practice or testing of the invention, the preferred methods and materials are described below. The materials, methods and examples are illustrative only, and are not intended to be limiting. Other features and advantages of the invention will be apparent from the detailed description and from the claims.

### DETAILED DESCRIPTION OF THE INVENTION

The invention provides a method for validation of cancer therapeutic targets in vivo, using inducible shRNAs and tumor xenografts. The inducible shRNA method operates as an in vivo RNAi competition assay. The competition is among subpopulations of tumor cells, where each subpopulation expresses a different shRNA targeting a different gene. Upon induction of shRNA expression, which is essentially simultaneous in all the subpopulations, expression of the gene of interest, i.e., the target gene, in each subpopulation is suppressed. If the target gene in a given tumor cell subpopulation helps that subpopulation to survive and proliferate in the tumor, i.e., is necessary for tumorigenicity, the cells of that subpopulation will be at a selective disadvantage with respect to the other tumor cell subpopulations, when shRNA expression is induced. Consequently, over time, the representation of that subpopulation will diminish relative to the other subpopulations, and eventually will disappear from the tumor, in the presence of the inducer. Conversely, if the target gene conferred on that subpopulation a competitive disadvantage, the representation of that subpopulation will increase, upon induction of shRNA expression. The third possibility is that the target gene is selectively neutral. In that case, the relative abundance of the target gene will not change significantly upon induction of shRNA expression.

Interfering RNAs targeted against any gene of interest can be designed routinely, e.g., using publicly available software such as OligoEngine™ (www.oligoengine.com). Alternatively, shRNAs, including shRNA cloned into suitable expression vectors, are commercially available from various commercial vendors, e.g., OriGene, Rockville, Md.; Open Biosystems, Huntsville, Ala.; BD Biosystems, San Jose, Calif.; and ExpressOn Biosystems Ltd., Midlothian, UK.

Intracellular transcription of dsRNAs can be achieved by cloning the dsRNA-encoding sequences into RNA polymerase III (Pol III) transcription units, which normally encode the small nuclear RNA U6 or the human RNAse P RNA H1. The dsRNA also can be cloned into RNA polymerase I or polymerase II transcription units or various other promoters. In general, there are two alternatives for producing the desired siRNA in situ. The sense and antisense strands of the siRNA duplex can be transcribed from separate promoters, or they can be expressed as fold-back step-loop structure that gives rise to a double stranded siRNA after intracellular processing.

Either way, expression of the dsRNAs is controlled under an inducible system. Several useful, well-characterized inducible expression systems are known and commercially available in whole or in part. Examples of suitable inducible systems include, but are not limited to, the tetracycline repressor system (Invitrogen, Carlsbad, Calif.), the Tet on/off system (BD Bioscience, San Jose, Calif.), the lac operator-repressor system (Stratagene, LaJolla, Calif.) and the Cre-Lox system (DuPont, Wilmington, Del.).

For further information on design and expression of shRNA vectors, see generally, Brummelkamp et al., 2002, Science 296:550-553; Paddison et al., 2002, Cancer Cell 2:17-23; Paul et al., 2002, Nat. Biotech. 20:505-508; Sui et al., 2002, Proc. Natl. Acad. Sci. USA, 99:5515-5520; Paddison, 2002, Genes Dev. 16:948-958; Gupta et al., 2004, Proc. Nat. Acad. Sci. USA 101:1927-1932.

Any type of human tumor cell line that can be grown as a xenograft in an immunocompromised mouse can be tested in this system. For example, any of the NCI 60 cell lines would be suitable for use in the invention. Preferred human cell lines include: HCT-116 colorectal carcinoma, DLD-1, HT-1080 fibrosarcoma, HCT-15 colon adenocarcinoma, A-549 lung carcinoma, SW 620 colorectal adenocarcinoma, LNCAP prostate carcinoma, 22Rv1 prostate carcinoma, DU145 prostate carcinoma metastasis to brain, PC-3 prostate adenocarcinoma.

To avoid transplant rejection, the tumor cells must be xenografted into immunocompromised mice. Mice homozygous for the severe combined immune deficiency spontaneous mutation (Prkdc^{scid}) commonly referred to as SCID mice, are preferred in practicing the invention. However, other types of immunocompromised mice, e.g., nude mice, may be used, as well. Numerous strains of useful immunocompromised mice are commercially available from sources such as The Jackson Laboratory (Bar Harbor, Me.) and Charles River Laboratories, Inc. (Wilmington, Mass.).

Because the methods of the present invention involve determining relative sizes of subpopulations of xenografted tumor cells expressing shRNAs targeting different genes, tumor cells representing at least two different shRNAs must be injected into a host mouse. Preferably, a negative control shRNA is included. An advantage offered by the present invention is that it allows the simultaneous testing of multiple, e.g., 2, 5, 10, 20 or 40, target candidate genes in the same tumor in the same mouse. In principle, the only limit on the number of genes that can be tested at once is the ability to discriminate “signal” from “noise” in the DNA preparation and quantitative PCR process.

As in other types of tumor xenograft experiments, there is considerable latitude in the total number of cells injected into the host mouse. The total number of cells injected can be optimized by a person skilled in the art, based on parameters such as tumor cell type, size of tumor desired, time until tumor harvest, and number of tumor cell subpopulations being tested.

It is not necessary for all the tumor cell subpopulations to be of equal size (equal numbers of cells) at the time of injection, but it is necessary for the relative sizes of the subpopulations to be known. Preferably, the subpopulations are of equal size, simply as a matter of convenience. Preferably, all the cells to be injected (all the subpopulations) are mixed to form the starting total population, prior to injection into the mouse. For injection, the cells can be in culture medium or any other medium that is nontoxic to the tumor cells and nontoxic to the host mouse when injected. Preferably, however, the cells are in a medium that includes some type of pharmaceutically acceptable polymer matrix to help the cells coalesce during and after the injection process. Matrigel™ brand (BD Biosciences, San Jose, Calif.) solubilized basement membrane preparation has been found suitable for this purpose.

After tumors have formed, induction of shRNA expression is initiated and maintained in the expression group, but not in the uninduced group. Once tumors have formed, the timing of the induction is not critical. A convenient practice is to induce shRNA expression when tumors are palpable, e.g., 5, 6, 7 or 8 days after injection of the tumor cells. Preferably, the size of the tumors at harvest is 500-3000 mm³. More preferably, the size of the tumors at harvest is 1000-2000 mm³. Timing of the tumor harvest can vary. Typically, the timing of tumor harvest is 15 to 40 days after injection of the tumor cells. In some embodiments of the invention, the timing is 20 to 30 days after injection of the tumor cells.

Quantitative assessment of the relative numbers of cells in each of the subpopulations following harvest of the tumors can be by any suitable method. Quantitative PCR is preferred. The design, synthesis and use of PCR primers for a given vector and set of shRNA coding sequences can be carried out readily by a person skilled in the art using conventional methods and commercially available equipment and reagents.

The invention is further illustrated by the following examples. The examples are provided for illustrative purposes only, and are not to be construed as limiting the scope or content of the invention in any way.

### EXAMPLE 1

### Vector Construction and Cell Transfection

Lentiviral vectors encoding interfering dsRNA against the target genes were generated. To target specific regions of a target gene mRNA, complements of the selected nucleotides were used with a primer specific to the U6 small RNA promoter to form double-stranded DNA in a polymer chain reaction, using a vector containing this U6 promoter as a template. The PCR product was then ligated into a pENTR11 vector (Invitrogen, Carlsbad, Calif.) into which the inducible U6TO2B promoter (described in WO 2004/056964) was cloned. Expression of the insert resulted in expression of a short hairpin RNA.

The U6TO2B-[target gene] shRNA expression cassettes were shuttled into the pLenti6 lentiviral vector using LR Clonase (Invitrogen cat. Nos.V496-10 and 11791-019). Lentiviruses were generated using Invitrogen's packaging system. In that process, 6 μg of lentiviral DNA was mixed with 6 μg of packaging mix from the ViraPower™ lentiviral support kit (Invitrogen cat. No. 11668-027). Supernatants were harvested after 48 hours, and viral titers were estimated by performing infections of HCT-116 cells with serial dilutions.

The shRNA-encoding lentiviral vectors were introduced into a human colorectal cell line HCT-116 stably expressing luciferase, a hygromycin resistance gene, and TetR (as described in WO 2004/056964). Briefly, the HCT-116 cell line was engineered as follows. HCT-116 cells were infected with a retrovirus expressing luciferase and the hygromycin resistance gene. After selection on hygromycin (50 μg/ml), the cells were infected with a lentivirus expressing a codon-optimized version of TetR (gpTetR), under the control of the PGK promoter. The gpTetR expression construct contained a Zeocin resistance gene. The stably transfected cells were selected with Zeocin (50 μg/ml) and then subcloned. Single clones were tested for adequate gpTetR expression, using real time RT-PCR. Selected clones were subsequently infected with lentiviral vectors containing target gene shRNAs under the control of the inducible promoter U6TO2B, or no shRNA (empty vector).

### EXAMPLE 2

### Varying Signal-to-Noise Ratio

HCT-116/Tet repressor (TetR) cells were engineered by lentiviral infection to express shRNAs against K-Ras, a gene previously reported to be essential for the oncogenic potential of HCT-116 cells. For use as a negative control, HCT-116/TetR cells expressing shRNAs against a control gene, luciferase, were also produced. The primary objective in this preliminary experiment was to test the ability of the assay to detect small subpopulations of the K-Ras shRNA-expressing cells against a relatively large background of other shRNA-expressing tumor cells.

The two types of engineered cells were cultured in vitro separately in DMEM with 10% Fetal Bovine Serum, and then harvested and mixed in different ratios (K-Ras shRNA expressing cells: luciferase shRNA expressing cells=1:3, 1:10, 1:30, and 1:100). The mixed populations of cells were injected into 40 SCID mice (10⁶ cells per injection, two injection sites per mouse). The mice were then divided into two groups, i.e., an shRNA expression group and an uninduced group. The shRNA expression group received doxycycline (2 μg/ml) administered orally in drinking water beginning at day six, to induce the expression of the shRNAs. The uninduced group did not receive doxycycline.

The tumors were collected at day 26. The genomic DNA was extracted using a commercial reagent kit according to the vendor's recommendations (Qiagen, Valencia, Calif.). Quantitative PCR with SYBR green (Qiagen) was used to determine the relative abundance of DNA encoding the shRNA hairpins, using a vector specific primer (pLentiTO2B_{—}1, CTCGACGGTATCGCTAGTCC) (SEQ ID NO: 1) and a hairpin-specific primer (K-Ras shRNA1 TGTATCGTCAAGGCATTGGT) (SEQ ID NO: 2) or Luciferase shRNA GCTCTCGCTGAGTTGGAATC) (SEQ ID NO: 3). During the quantitative PCR cycle, the polymerase was first activated at 95° C. for 15 minutes followed by 40 cycles of denaturation at 95° C., annealing at 54° C. and elongation at 72° C. The representation of the cells expressing each individual shRNA in the tumor samples was then calculated.

In this experiment, the tumor cell subpopulation expressing shRNAs against K-Ras was rapidly depleted from the mixed population of tumor cells after the administration of doxycycline, and resulting induction of shRNA expression. Although near the limit of detection, the presence of the HCT-116/TetR cells expressing the K-Ras hairpin was detectable in the doxycycline-treated tumors arising from the 1:100 mixed population of cells.

TABLE 1

| HCT116/TetR cells | % elimin. of K-Ras | % elim. of luciferase |
|---|---|---|
| expressing K-Ras | shRNA-expressing | shRNA-expressing |
| shRNA:Luciferase shRNA | cells | cells |
| 1:3 | 90.4 | 8.6 |
| 1:10 | 70.7 | 0.81 |
| 1:30 | 52.8 | −6.4 |
| 1:100 | 100 | −43.8 |

These results indicated that the assay is sufficiently sensitive to detect the depletion of a subpopulation of cells even when that particular subpopulation constitutes as little as 1% of the total tumor cell population.

### EXAMPLE 3

### Depletion of Multiple Subpopulations

HCT-116/Tet repressor cells were engineered as described above to express shRNAs against the genes listed in Table 2 below. The cells were cultured in vitro separately in DMEM with 10% fetal bovine serum, and harvested. Equal numbers of cells representing each shRNA were mixed to form the mixed population. The mixed population of cells was injected into 20 SCID mice (10⁶ cells per injection, two injection sites per mouse). The mice were then divided into two groups. One group (test group) received doxycycline in its drinking water beginning at day six, to induce the expression of the shRNAs. The other group (control group) received drinking water without doxycycline. The tumors were harvested at day 26.

The average weight of the tumors from mice not receiving doxycycline was 0.236 g (n=8). The average weight of tumors from mice receiving doxycycline was 0.212 g (n=11). Upon pulverization of the tumors, the DNA was extracted (Qiagen genomic DNA extraction kit) followed by quantitative PCR with SYBR green (Qiagen) to examine the abundance of the shRNA hairpins using a vector specific primer (pLentiTO2B_{—}1, CTCGACGGTATCGCTAGTCC) (SEQ ID NO: 1) and a hairpin-specific primer (listed in Table 2). During the quantitative PCR cycle, the polymerase was first activated at 95° C. for 15 minutes followed by 40 cycles of denaturation at 95° C., annealing at 54° C. and elongation at 72° C. The representation of the cells expressing each individual shRNA in the tumor samples was then calculated.

TABLE 2

| | | | | |
|---|---|---|---|---|
| | mRNA | % elimin. | | |
| Target | knock- | of | | |
| gene | down | subpop. | P value | Primer Sequence |

| | | | | |
|---|---|---|---|---|
| Trans- | Yes | −5.8 | 0.69 | GGAAGATTAGTTCTGACTTGG |
| ketolase | | | | (SEQ ID NO: 4) |
| Trans- | No | −13.5 | 0.86 | GGATGCTATTGCGCAGGCTG |
| ketolase | | | | (SEQ ID NO: 5) |
| K-Ras | Yes | 96.8 | 3.2 × 10⁻⁵ | GCGATATAGCTAGTTCAGGAT |
| | | | | (SEQ ID NO: 6) |
| K-Ras | No | −22.5 | 0.73 | GGATAGCCAACAATAGAGGTAAA |
| | | | | (SEQ ID NO: 7) |
| Ceramide | Yes | −187.2 | 5.1 × 10⁻⁵ | ATAGTACGCTCCTTCGCTATTC |
| Kinase | | | | (SEQ ID NO: 8) |
| Ramp2 | Yes | 99.4 | 2.0 × 10⁻⁷ | GTGAGTCTCAAAGATGATCC |
| (Hairpin 1) | | | | (SEQ ID NO: 9) |
| Ramp2 | Yes | 99.0 | 2.0 × 10⁻⁷ | ACTGTCTTTACTCCTCCATAC |
| (Hairpin 2) | | | | (SEQ ID NO: 10) |
| PTK7 | Yes | 95.8 | 1.9 × 10⁻⁴ | CTTGATGTTGCAGCTGTTGC |
| (Hairpin 1) | | | | (SEQ ID NO: 11) |
| PTK7 | Yes | 88.0 | 7.9 × 10⁻⁶ | CACTTTCAGCAATATTGGCC |
| (Hairpin 2) | | | | (SEQ ID NO: 12) |
| Luciferase | Yes | −323.6 | 0.026 | GCTCTCGCTGAGTTGGAATC |
| | | | | (SEQ ID NO: 3) |

In this experiment, HCT-116 cells with active shRNA against K-Ras were depleted by more than 96% from the mixed population of tumor cells after the administration of doxycycline in vivo. A similar result was not obtained with the mice receiving an inactive shRNA against K-Ras. In the same experiment, the average abundance of the cells expressing PTK-7 hairpin 1 or 2 was decreased by 95.8% or 88% respectively in tumors exposed to doxycycline compared to that in untreated tumors, suggesting PTK7 has an essential role in tumor viability or maintenance. Similarly, the average abundance of the cells expressing Ramp2 hairpin 1 or 2 was decreased by more than 99% in tumors treated with doxycycline, suggesting Ramp2 also has an essential role in tumor viability or maintenance.

### EXAMPLE 4

### Depletion of Multiple Subpopulations

HCT-116/Tet repressor cells individually expressing a dozen shRNAs against genes of interest were cultured in vitro separately and then mixed in equal ratio at concentration of 10⁷ cells/ml. The mixed population of cells was mixed with Matrigel (1:1) and injected into 20 SCID mice (10⁶ cells/0.2 mls per injection, 2 injection sites per mouse). The mice were then divided into two groups. One group (shRNA expression group) received doxycycline at day six to induce the expression of shRNAs, and the other group (uninduced group) did not. The tumors were collected at day 21.

The average weight of the tumors from mice not receiving doxycycline treatment was 0.329 g (n=28) and the average weight of tumors from mice receiving doxycycline treatment was 0.356 g (n=27). Upon pulverization of the tumors, the DNA was extracted (Qiagen). Equal amounts of DNA from tumors with or without doxycycline treatment were pooled to provide samples, i.e., an induced sample and a noninduced sample for each shRNA. This was in contrast to Example 3 (above) in which DNA from each tumor was processed separately.

Quantitative PCR with SYBR green (Qiagen) was used to examine the abundance of genomic DNA encoding each of the shRNAs using a vector specific primer, pLentiTO2B_{—}1 (CTCGACGGTATCGCTAGTCC) (SEQ ID NO: 1), and a hairpin-specific primer (listed in Table 3) in the two groups of DNA. During the PCR cycle, the polymerase was first activated at 95° C. for 15 minutes followed by 40 cycles of denaturation at 95° C., annealing at 54° C. and elongation at 72° C. The representation of the cells expressing each individual shRNA in the tumor samples was then calculated.

TABLE 3

| | | | | |
|---|---|---|---|---|
| | mRNA | % elim. | | |
| Target | knock- | of | | |
| Gene | down | subpop. | P value | Primer Sequence |

| | | | | |
|---|---|---|---|---|
| c17orf26 | Yes | 98.6 | 6.9 × 10⁻⁴ | CCGTAGAGCCGAAGTTCAGT |
| | | | | (SEQ ID NO: 13) |
| HAS3 | Yes | 78.1 | 1.3 × 10⁻⁷ | GAGAATGTTCCAGATGCGGC |
| | | | | (SEQ ID NO: 14) |
| G6PD + | Yes | 19.8 | 0.065 | GATGTCGGATGCACACATATTA |
| Trans- | | | | (SEQ ID NO: 15) |
| ketolase | | | | |
| Trans- | Yes | −72.2 | 5.3 × 10⁻⁶ | GGAAGATTAGTTCTGACTTGG |
| ketolase | | | | (SEQ ID NO: 4) |
| Trans- | No | 7.2 | 0.025 | GGATGCTATTGCGCAGGCTG |
| ketolase | | | | (SEQ ID NO: 5) |
| K-Ras | Yes | 91.1 | 8.3 × 10⁻⁵ | GCGATATAGCTAGTTCAGGAT |
| | | | | (SEQ ID NO: 6) |
| K-Ras | No | 23.5 | 0.23 | GGATAGCCAACAATAGAGGTAAA |
| | | | | (SEQ ID NO: 7) |
| Ramp2 | Yes | 98.1 | 3.3 × 10⁻⁶ | GTGAGTCTCAAAGATGATCC |
| (Hairpin 1) | | | | (SEQ ID NO: 9) |
| Ramp2 | Yes | 99.9 | 8.3 × 10⁻⁶ | ACTGTCTTTACTCCTCCATAC |
| (Hairpin 2) | | | | (SEQ ID NO: 10) |
| PTK7 | Yes | 92.6 | 3.1 × 10⁻⁶ | CTTGATGTTGCAGCTGTTGC |
| (Hairpin 1) | | | | (SEQ ID NO: 11) |
| PTK7 | Yes | 58.1 | 1.7 × 10⁻⁷ | CACTTTCAGCAATATTGGCC |
| (Hairpin 2) | | | | (SEQ ID NO: 12) |
| Luciferase | Yes | −44.7 | 0.42 | GCTCTCGCTGAGTTGGAATC |
| | | | | (SEQ ID NO: 3) |

In this experiment, the positive control, a subpopulation expressing an active shRNA against K-Ras was depleted by more than 91% from the mixed population of tumor cells in the presence of doxycycline. In contrast, there was no depletion of the negative control subpopulation expressing a known inactive shRNA against K-Ras. As in previous experiments, the subpopulations of cells inducibly expressing shRNAs targeted against PTK-7 and Ramp2 were rapidly depleted in the presence of doxycycline. In addition, subpopulations expressing shRNAs suppressing expression of HAS3 or c17orf26 were depleted. This indicated a possible role of the HAS3 and c17orf26 genes in tumor maintenance.

Other embodiments are within the following claims.

What is claimed is:

1. An in vivo method of determining whether a gene of interest is necessary for a tumor cell to maintain its proliferation and survival properties, the method comprising:
(a) providing a first subpopulation of cells of a given tumor-forming cell line, wherein the subpopulation is engineered to express an shRNA against a first gene of interest, in response to an inducer;
(b) providing one or more additional subpopulations of cells of the same cell line, wherein each subpopulation is engineered to express an shRNA in response to the inducer;
(c) injecting into each of at least two immunocompromised mice a mixture of cells representing the first subpopulation of cells and each of the one or more additional subpopulations of cells;
(d) allowing time for tumors to develop in the mice from the injected cells;
(e) administering an effective amount of the inducer to at least one mouse, thereby establishing an shRNA expression group, while withholding the inducer from at least one mouse, thereby establishing an uninduced group;
(f) harvesting the tumors after a suitable time period;
(g) determining the relative representation of the cells engineered to express the shRNA against each gene of interest in the shRNA expression group and in the uninduced group.

2. The method of claim 1, wherein the tumor-forming cell line is a human cell line.

3. The method of claim 2, wherein the tumor-forming cell line is selected from the group consisting of HCT-116, DLD-1, HT-1080, HCT-15, A-549, SW 620, LNCAP, 22Rv1, DU145, and PC-3.

4. The method of claim 1, wherein the one or more additional subpopulations of cells comprise a subpopulation of cells engineered to express at least one control shRNA.

5. The method of claim 4, wherein the control shRNA is a negative control.

6. The method of claim 4, wherein the control shRNA is a positive control.

7. The method of claim 1, wherein the one or more additional subpopulations of cells comprise at least one subpopulation of cells engineered to express an shRNA against at least one additional gene of interest.

8. The method of claim 7, wherein the one or more additional subpopulations of cells comprise at least one subpopulation of cells engineered to express an shRNA against at least two additional genes of interest.

9. The method of claim 1, wherein the one or more additional subpopulations of cells comprise at least one subpopulation of cells engineered to express an shRNA against at least 5 additional genes of interest.

10. The method of claim 1, wherein the one or more additional subpopulations of cells comprise at least one subpopulation of cells engineered to express an shRNA against at least 10 additional genes of interest.

11. The method of claim 1, wherein the mixture of the first subpopulation of cells and the one or more additional subpopulations of cells injected into each mouse consists of 10³ to 10⁸ cells.

12. The method of claim 11, wherein the mixture of the first subpopulation of cells and the one or more additional subpopulations of cells injected into each mouse consists of 10⁵ to 10⁷ cells.

13. The method of claim 12, wherein the mixture of the first subpopulation of cells and the one or more additional subpopulations of cells injected into each mouse consists of approximately 10⁶ cells.

14. The method of claim 1, wherein the determining the relative representation of the cells is measured by quantitative PCR analysis.


SEQUENCE LISTING (15 sequences)

SEQ ID NO: 1 - DNA, 20 bp, Artificial Sequence
  Description of Artificial Sequence Synthetic primer
ctcgacggtatcgctagtcc

SEQ ID NO: 2 - DNA, 20 bp, Artificial Sequence
  Description of Artificial Sequence Synthetic primer
tgtatcgtcaaggcattggt

SEQ ID NO: 3 - DNA, 20 bp, Artificial Sequence
  Description of Artificial Sequence Synthetic primer
gctctcgctgagttggaatc

SEQ ID NO: 4 - DNA, 21 bp, Artificial Sequence
  Description of Artificial Sequence Synthetic primer
ggaagattagttctgacttgg

SEQ ID NO: 5 - DNA, 20 bp, Artificial Sequence
  Description of Artificial Sequence Synthetic primer
ggatgctattgcgcaggctg

SEQ ID NO: 6 - DNA, 21 bp, Artificial Sequence
  Description of Artificial Sequence Synthetic primer
gcgatatagctagttcaggat

SEQ ID NO: 7 - DNA, 23 bp, Artificial Sequence
  Description of Artificial Sequence Synthetic primer
ggatagccaacaatagaggtaaa

SEQ ID NO: 8 - DNA, 22 bp, Artificial Sequence
  Description of Artificial Sequence Synthetic primer
atagtacgctccttcgctattc

SEQ ID NO: 9 - DNA, 20 bp, Artificial Sequence
  Description of Artificial Sequence Synthetic primer
gtgagtctcaaagatgatcc

SEQ ID NO: 10 - DNA, 21 bp, Artificial Sequence
  Description of Artificial Sequence Synthetic primer
actgtctttactcctccatac

SEQ ID NO: 11 - DNA, 20 bp, Artificial Sequence
  Description of Artificial Sequence Synthetic primer
cttgatgttgcagctgttgc

SEQ ID NO: 12 - DNA, 20 bp, Artificial Sequence
  Description of Artificial Sequence Synthetic primer
cactttcagcaatattggcc

SEQ ID NO: 13 - DNA, 20 bp, Artificial Sequence
  Description of Artificial Sequence Synthetic primer
ccgtagagccgaagttcagt

SEQ ID NO: 14 - DNA, 20 bp, Artificial Sequence
  Description of Artificial Sequence Synthetic primer
gagaatgttccagatgcggc

SEQ ID NO: 15 - DNA, 22 bp, Artificial Sequence
  Description of Artificial Sequence Synthetic primer
gatgtcggatgcacacatatta